(19) United States
(12) Patent Application Publication
(10) Pub. No.: US 2009/0176233 A1
(43) Pub. Date: Jul. 9, 2009

(54) Screening assays for polymerase enhancement

(75) Inventors: Sonya Clark, Oakland, CA (US); Homero L. Rey, Campbell, CA (US); Fred Christians, Los Altos Hills, CA (US); Jonas Korlach, Newark, CA (US)

Correspondence Address: QUINE INTELLECTUAL PROPERTY LAW GROUP, P.C., P O BOX 458, ALAMEDA, CA, 94501, US

(73) Assignee: Pacific Biosciences of California, Inc., Menlo Park, CA (US)

(21) Appl. No.: 12/315,844

(22) Filed: Dec. 5, 2008

Related U.S. Application Data
(60) Provisional application No. 61/005,631, filed on Dec. 5, 2007.

(51) Int. Cl. C12Q 1/68; C12N 9/00; C12M 1/00

(52) U.S. Cl. 435/6; 435/183; 435/287.2

(57) ABSTRACT

Methods of screening for and selecting for improved polymerases suitable for single molecule sequencing are provided.

### CROSS-REFERENCE TO RELATED APPLICATIONS

This application claims priority to and benefit of U.S. Ser. No. 61/005,631, filed Dec. 5, 2007, entitled “Screening Assays for Polymerase Enhancement” by Sonya Clark, et al., which is incorporated herein by reference in its entirety for all purposes.

### STATEMENT AS TO RIGHTS TO INVENTIONS MADE UNDER FEDERALLY SPONSORED RESEARCH AND DEVELOPMENT

Portions of the invention were made with government support under NHGRI Grant No. R01HG003710. The government has certain rights to the invention.

### FIELD OF THE INVENTION

The invention relates to identification of polymerases having improved features for use with single molecule sequencing. Methods of selecting and screening groups of polymerases as well as for tracking and cataloging particular polymerases are described.

### BACKGROUND OF THE INVENTION

DNA polymerases are instrumental in the core function of replicating the genomes of living organisms. In addition to this central role in biology, however, DNA polymerases are also ubiquitous tools of biotechnology. For example, they are widely used for reverse transcription, amplification, labeling, sequencing, etc. Such uses are central technologies for a variety of biotechnology applications such as sequencing, nucleic acid amplification, cloning, protein engineering, diagnostics, molecular medicine and many other technologies.

Because of the significance of DNA polymerases, they have been extensively studied. Crystal structures have been determined for many polymerases, which often share a similar architecture, and the basic mechanisms of action for many polymerases have been determined. The study of polymerases has primarily focused on phylogenetic relationships among polymerases, structure of polymerases, structure-function features of polymerases, and the role of polymerases in DNA replication and other basic biology, as well as ways of using DNA polymerases in biotechnology. For a review of polymerases, see, e.g., Hübscher, et al. (2002) EUKARYOTIC DNA POLYMERASES Annual Review of Biochemistry 71:133-163; Alba (2001) “Protein Family Review: Replicative DNA Polymerases” Genome Biology 2(1): Reviews 3002.1-3002.4; Steitz (1999) “DNA polymerases: structural diversity and common mechanisms” J. Biol. Chem. 274:17395-17398 and Burgers, et al. (2001) “Eukaryotic DNA polymerases: proposal for a revised nomenclature” J. Biol. Chem. 276(47):43487-90.

One useful application of polymerases in biotechnology includes various permutations of nucleic acid sequencing. For example, zero-mode waveguide (ZMW) sequencing, as well as other single molecule sequencing procedures utilize polymerases.

While various DNA polymerase mutants/variants have been isolated and/or identified that have altered functions, e.g., nucleotide analogue incorporation relative to wild-type counterpart enzymes, particular functionality is often desired with a given polymerase application.

Thus, the ability to improve specificity, processivity, or other features of DNA polymerases to match particular applications, especially in regard to nucleic acid sequencing by incorporation applications, would be highly desirable in a variety of contexts. The present invention provides methods to screen and select for new DNA polymerases with modified properties useful for nucleic acid sequencing applications, and particularly for use in sequencing by incorporation applications, as well as many other features that will become apparent upon a complete review of the following.

### SUMMARY OF THE INVENTION

The present invention comprises, inter alia, methods of identifying improved nucleic acid polymerases (e.g., those having one or more improved characteristics useful or desirable for single molecule sequencing such as, but not limited to, single molecule sequencing in zero mode waveguides). In such methods, one or more polymerases (e.g., a number of randomly mutated, rationally mutated/designed, or otherwise potentially improved polymerases) are provided to be screened/selected for; such polymerases are screened and/or selected for the desired improved characteristic(s); and the improved polymerases are identified based on the results of such screening and/or selecting. In such methods, the one or more improved characteristics of the polymerase can comprise: increased fluorophore-dependent photostability; increased fluorophore-independent photostability; increased residence time; increased affinity; use of nontraditional divalent cations; decreased cognate nucleotide disassociation or “branching” activity; increased fidelity; and decreased exonuclease activity. Also in such methods, the selecting and/or screening can include one or more of: polymerase extension activity in the presence of a fluorescently labeled nucleotide and light; polymerase extension activity in the absence of a fluorescently labeled nucleotide and light; rate of incorporation of a marker nucleotide by a polymerase; incorporation of a marker nucleotide by polymerase extension activity under limiting concentrations of nucleotides; rate of incorporation of a marker nucleotide by a polymerase in the presence of nontraditional divalent cations; rate of cognate nucleotide disassociation (branching); and removal of a marker nucleotide from a nucleic acid by a polymerase.

In various embodiments of the methods herein, the potentially improved polymerases (i.e., the polymerases that are screened/selected for by the invention) are randomly mutated nucleic acid polymerases. It will be appreciated that the particular mutation format or procedure used to generate the mutated nucleic acid polymerases should not necessarily be taken as limiting and can include, but is not limited to, e.g., overlap extension PCR to recombine fragments of homologous genes, random point mutagenesis by error-prone PCR, mutagenesis by total gene synthesis, site-directed point mutagenesis, saturation mutagenesis of one or more specific residues, and simultaneous combinatorial saturation mutagenesis of multiple specific residues. Furthermore, mutants are optionally generated through rationally designed mutation such as structure-function modeling to generate site-specific mutations either individually or in combinations, or homology modeling and recombination of several related polymerases.

The polymerases to be screen/selected herein can optionally be selected/screened for viable polymerase activity either before or simultaneous with the screens/selections for the improved characteristics.

In particular embodiments herein, the improved characteristic being screened/selected for can be increased fluorophore-dependent photostability, while the screening and/or selecting is polymerase extension activity in the presence of a fluorescently labeled nucleotide and light. In some such embodiments, the invention further comprises providing one or more fluorophore-labeled oligonucleotide that is hybridized (or that is capable of hybridizing) to a nucleic acid template to be acted upon by the polymerases being screened/selected. In some embodiments, the fluorophore is located at the 3′ end, or near the 3′ end, of the oligonucleotide. Also, in some embodiments, the fluorophore is in close proximity to the binding site of the polymerase where the polymerase interacts with the nucleic acid template.

In other embodiments, the improved characteristic comprises increased fluorophore-independent photostability and the screening and/or selecting comprises polymerase extension activity in the presence of light, but in the absence of a fluorescently labeled nucleotide.

In yet other embodiments, the improved characteristic comprises increased residence time and the screening and/or selecting comprises rate of incorporation of a marker nucleotide by a polymerase.

Still other embodiments include wherein the improved characteristic comprises increased affinity and the screening and/or selecting comprises incorporation of a marker nucleotide by polymerase extension activity under limiting concentrations of nucleotides. In some embodiments the increased or altered affinity is for a nucleotide, a normative nucleotide, or nucleotide analogue.

In other embodiments the improved characteristic comprises use of nontraditional divalent cations and the screening and/or selecting comprises rate of incorporation of a marker nucleotide by a polymerase in the presence of nontraditional divalent cations.

In other embodiments the improved characteristic comprises decreased cognate nucleotide disassociation activity and the screening and/or selecting comprises rate of cognate nucleotide disassociation.

In other embodiments the improved characteristic comprises increased fidelity and the screening and/or selecting comprises rate of incorporation of a non-cognate nucleotide.

In still further embodiments the improved characteristic comprises decreased exonuclease activity and the screening and/or selecting comprises removal of a marker nucleotide or exposure/activation of a marker nucleotide from a nucleic acid by a polymerase.

In the various embodiments herein, the polymerase activity of the one or more potentially improved polymerases and/or the improved polymerase can be determined by oligonucleotide probe hybridization. Also in the various embodiments, the one or more potentially improved polymerases and/or the improved polymerase can be tracked by DNA identification tagging.

In the various embodiments herein, the improved characteristic(s) that are screened/selected for can be identified simultaneously or sequentially. In other words, in some embodiments a first characteristic is screened for in a first iteration and a second characteristic is screened for in a second iteration; while in other embodiments, the two characteristics are screened for at the same time in the same iteration. It will be appreciated that in the various embodiments, the characteristics can be the same or different in each round of identification and that the screens/selections can be the same or different in each round of identification. Additionally, it will also be appreciated that various embodiments can comprise numerous rounds or iterations (e.g., 2, 3, 4, 5, 6, 7, 8, 9, 10, 15, 20, 25, 50, 100, 500 or more) of screening/selecting for the one or more characteristics. Also, the polymerases being screened/selected can undergo either random and/or rationally designed alteration/mutation between screening/selection iterations.

In other aspects, the invention includes polymerase(s) identified by the methods herein.

In other aspects, the invention comprises a system for identifying putative improved polymerases. Such system comprises a screening module configured to perform one or more of: polymerase extension activity in the presence of a fluorescently labeled nucleotide and light; polymerase extension activity in the absence of a fluorescently labeled nucleotide and light; incorporation of a marker nucleotide by a polymerase; incorporation of a marker nucleotide by polymerase extension activity under limiting concentrations of nucleotides; incorporation of a marker nucleotide by a polymerase in the presence of nontraditional divalent cations; extensions wherein cognate nucleotide disassociation or branching could optionally occur; and removal of a marker nucleotide from a nucleic acid by a polymerase. Such system also comprises a detector configured to detect one or more improved characteristic selected from: increased fluorophore-dependent photostability; increased fluorophore-independent photostability; increased residence time; increased affinity; use of nontraditional divalent cations; decreased (or increased) cognate nucleotide disassociation activity; increased fidelity; and decreased exonuclease activity. The improved characteristics can be tracked by, e.g., determining/following the rate of incorporation of a marker nucleotide(s) under various reaction conditions, rate of cognate nucleotide disassociation, etc.

These and other objects and features of the invention will become more fully apparent when the following detailed description is read in conjunction with the accompanying figures.

### BRIEF DESCRIPTION OF THE DRAWINGS

FIG. 1 shows a generalized overview of exemplary screening/selection methods of the invention.

FIG. 2 shows a schematic of an exemplary screen for residence time.

FIG. 3a shows a schematic of an exemplary screen for CND or “branching” fraction.

FIG. 3b shows a graph illustrating the differences in polymerase half-life of nonscreened/nonselected polymerases and polymerases that have been screened/selected with various methods herein.

FIG. 4 shows an illustration of an exemplary DNA ID tag on an enzyme and an associated polymerase.

FIG. 5 illustrates the structure of an oligonucleotide with a dye (Oregon Green 488, an amino dC dye) covalently tethered to the terminal base.

FIG. 6 shows a graph of the amount of noncognate sample (as a percentage of events) of selected polymerases that were screened through various methods of the invention.

### DETAILED DISCUSSION OF THE INVENTION

The current invention provides various screening/selecting methods (which are optionally used singularly or in any combination) to identify nucleic acid polymerases having desired properties. The desired properties typically comprise presence or absence of a trait, or an increase or decrease in a trait, as compared to the same trait in a “control” polymerase (e.g., one that is a wild type or nonmutated/nonrecombinant polymerase, or one that is a predecessor or parental polymerase to the enzyme being tested). As explained throughout, the polymerases identified through the methods herein are generally useful in a variety of contexts, and are particularly suited for use in nucleic acid sequencing applications which identify sequence information through monitoring of the polymerase mediated template dependent extension of primer sequences. In particularly preferred contexts, the screening/selecting processes described herein are used to identify polymerases that are optimized for performance in single molecule sequence by incorporation methods. Although primarily described in terms of, and for use in, such single molecule sequencing applications, it will be appreciated that the value of the screening/selecting processes described herein, and the enzymes that are identified through such, will not necessarily be limited to such particular applications.

In one example of sequencing by incorporation in which the polymerases identified through the current invention can be used, a polymerase reaction can be isolated within an extremely small observation volume that effectively results in observation of an individual polymerase molecule and its activity. Such small observation volumes can be achieved by immobilizing the polymerase enzyme within an optical confinement, such as a Zero Mode Waveguide (ZMW). For a description of ZMWs and their application in single molecule analyses, and particularly nucleic acid sequencing, see, e.g., Eid, et al., (2008) “Real-Time DNA Sequencing form Single Polymerase Molecules,” Science, in press (Science 20Nov. 2008:/162986v1/; DOI: 10.1126/Science.1162986), Levene, et al., (2003) “Zero-mode waveguides for single-molecule analysis at high concentrations, Science 299:682-686, Published U.S. Pat. Appl. No. 2003/0044781, and U.S. Pat. No. 6,917,726, each of which is incorporated herein by reference in its entirety for all purposes.

ZMW sequencing systems typically include a detector configured to detect a signal from the ZMW reaction chamber. Detection is usually performed by exciting the observation volume with an appropriate light source, such as a laser, and then detecting induced fluorescence with appropriate detection optics. Often, the excitation and detection optics are integrated (e.g., using an epi-fluorescent excitation/detection apparatus). Signals that are detected can be digitized and sent to a sequence assembly module that assembles the signals from sampling events into an overall sequence of the template nucleic acid.

It will be appreciated that polymerases used for nucleic acid sequencing applications, including those methods employing ZMWs, will be subjected to particular stresses and working conditions and that sequencing can typically be improved through proper optimization of the polymerase used. In particular, in the context of sequencing by incorporation, different methods or processes of sequencing may display different sensitivities to certain characteristics of the enzyme that is used in extending the primer sequence. For example, in most methods, the processivity, or ability of the enzyme to continue synthesizing long stretches of DNA, can directly impact the overall readlength of the process. This is of particular interest in single molecule systems that rely upon individual single polymerase/template/primer complexes. Similarly, for single molecule approaches, the fidelity of the overall system, the rate at which incorrect bases are incorporated, can directly impact the accuracy of the overall process, because every error is potentially identified as sequence information.

Other characteristics of polymerases that can impact their efficacy or desirability/suitability in sequence operations include the reaction kinetics of the enzyme toward the nucleotide or nucleotide analog used in the reaction. Such kinetics will impact the rate at which sequence information can be determined, the affinity of the polymerase toward desired reagents, and the like. Kinetics can include overall reaction rates for incorporation or can be broken down into the kinetics of the various stages of the incorporation reaction. Other enzymatic parameters that can impact various sequencing reactions include the residence time of the nucleotide or nucleotide analog in the active site of the polymerase (and by implication, for example, within an observation volume of an optical confinement); the sensitivity of a polymerase enzyme to adverse effects of prolonged illumination in the presence of photoactivatable species, e.g., fluorescent dyes; the tendency of a polymerase to bind a correct nucleotide without actually incorporating it into the primer extension reaction, also referred to as “cognate nucleotide disassociation” (CND) or, as “branching” or “stuttering.”

Therefore, the present invention provides methods to screen/select for polymerases that comprise particular characteristics, e.g., especially those suited to single molecule sequencing protocols. In general, the present invention comprises methods of screening and selecting one or more nucleic acid polymerase (e.g., from a pool or library of mutated polymerases) to identify such polymerases that have desirable characteristics for single molecule sequencing. It will be appreciated that the invention comprises a number of screening/selecting aspects, as well as methods to track and/or catalog particular enzymes and that each aspect can be used either by itself or in combination with any of the other aspects herein. Indeed, the screens/selections and other aspects of the invention can also be used in conjunction with other screens/selections not of the invention. Also, it will be appreciated that while the screenings are typically used to identify polymerases suitable for single molecule sequencing, the polymerase thus identified can be used in many other situations. The qualities of the polymerases that are identified will make them beneficial to other applications. Therefore, description of the identified enzymes' use in single molecule sequencing should not be taken as precluding their use (or the use of the screens/selections) in other applications.

### DNA Polymerases

A large number of polymerases of various types are well known and have been the subject of decades of focused research. DNA polymerases that can be screened/selected through use of the current invention and that can also be modified (either randomly or rationally) and then screened/selected with the invention, are generally available from any number of commercial sources and from numerous organisms and/or can be modified or generated through any of a number of ways.

DNA polymerases are typically classified into six main groups based upon various phylogenetic relationships, e.g., E. coli Pol I (class A), E. coli Pol II (class B), E. coli Pol III (class C), Euryarchaeotic Pol II (class D), human Pol beta (class X), and E. coli UmuC/DinB and eukaryotic RAD30/xeroderma pigmentosum variant (class Y). For a review of recent nomenclature, see, e.g., Burgers, et al. (2001) “Eukaryotic DNA polymerases: proposal for a revised nomenclature” J. Biol. Chem. 276(47):43487-90. For a review of polymerases, see, e.g., Hübscher, et al. (2002) EUKARYOTIC DNA POLYMERASES Annual Review of Biochemistry 71:133-163; Alba (2001) “Protein Family Review: Replicative DNA Polymerases” Genome Biology 2(1):reviews 3002.1-3002.4; and Steitz (1999) “DNA polymerases: structural diversity and common mechanisms” J. Biol. Chem. 274:17395-17398.

The basic mechanisms of action for many polymerases have been determined. The sequences of literally hundreds of polymerases are publicly available, and the crystal structures for many of these have been determined, or can be inferred based upon similarity to solved crystal structures for homologous polymerases. Furthermore, a variety of polymerases having characteristics adapted to single molecule sequencing reactions are known (see, e.g., Hanzel, et al. POLYMERASES FOR NUCLEOTIDE ANALOGUE INCORPORATION, WO 2007/076057; Hanzel, et al. ACTIVE SURFACE COUPLED POLYMERASES, WO 2007/075987; and Hanzel, et al. PROTEIN ENGINEERING STRATEGIES TO OPTIMIZE ACTIVITY OF SURFACE ATTACHED PROTEINS, WO 2007/075873). All of such polymerases can optionally be used as, or used to generate further, polymerases to be screened/selected herein.

Available DNA polymerase enzymes have been modified in any of a variety of ways, e.g., to reduce or eliminate exonuclease activities (many native DNA polymerases have a proof-reading exonuclease function that can interfere with sequencing applications), to simplify production by making protease digested enzyme fragments such as the Klenow fragment recombinant, etc. Again, any of these available polymerases, as well as many others, can be screened/selected for directly or can be modified in any of myriad ways and the resulting mutated/altered polymerases screened and/or selected through the methods of the instant invention.

As stated, many such polymerases that are suitable for screening/selection directly and/or for modification and subsequent screening/selection by the invention, are available commercially. For example, Human DNA Polymerase Beta is available from R&D systems. DNA polymerase I is available from Epicenter, GE Health Care, Invitrogen, New England Biolabs, Promega, Roche Applied Science, Sigma Aldrich and many others. The Klenow fragment of DNA Polymerase I is available in both recombinant and protease digested versions, from, e.g., Ambion, Chimerx, eEnzyme LLC, GE Health Care, Invitrogen, New England Biolabs, Promega, Roche Applied Science, Sigma Aldrich and many others. φ29 DNA polymerase is available from e.g., Epicenter. Poly A polymerase, reverse transcriptase, Sequenase, SP6 DNA polymerase, T4 DNA polymerase, T7 DNA polymerase, and a variety of thermostable DNA polymerases (Taq, hot start, titanium Taq, etc.) are available from a variety of the above and other sources. Other commercial DNA polymerases include Phusion™ High-Fidelity DNA Polymerase available from New England Biolabs; GoTaq® Flexi DNA Polymerase available from Promega; RepliPHI™ φ29 DNA Polymerase from EPICENTRE; PfuUltra™ Hotstart DNA Polymerase available from Stratagene; and KOD HiFi DNA Polymerase available from Novagen. A list of many commercially available polymerases can be found on the Internet at Biocompare.com.

In addition to commercial sources, polymerases to be screened/selected with the methods herein or to be randomly mutated and/or rationally designed to create polymerases to be screened/selected can also or alternatively be isolated from one or more organisms, e.g., eubacteria, archaebacteria, yeasts/fungi, eukaryotes (e.g., humans), etc.

### Libraries and Kinetics Involved in Screening/Selecting of Polymerases

In the instant invention, screening/selecting are used to determine whether a polymerase (e.g., a mutated polymerase) displays a modified activity for a particular activity as compared to another DNA polymerase (e.g., one that is wild type or non-mutated). For example, k_{cat}, Kₘ, Vₘₐₓ, or k_{cat}/Kₘ (or other activities as described herein) of a recombinant or other DNA polymerase can be determined as discussed herein. Those of skill in the art will be familiar with k_{cat}, Kₘ, Vₘₐₓ, or k_{cat}/Kₘ and other common enzymatic measurements and ways to determine them.

As is well known in the art, for enzymes obeying simple Michaelis-Menten kinetics, kinetic parameters are readily derived from rates of catalysis measured at different substrate concentrations. The Michaelis-Menten equation, V=Vₘₐₓ[S]([S]+Kₘ)⁻¹, relates the concentration of uncombined substrate ([S], approximated by the total substrate concentration), the maximal rate (Vₘₐₓ, attained when the enzyme is saturated with substrate), and the Michaelis constant (Kₘ, equal to the substrate concentration at which the reaction rate is half of its maximal value), to the reaction rate (V).

For many polymerase enzymes, Kₘ is equal to the dissociation constant of the enzyme-substrate complex and is thus a measure of the strength of the enzyme-substrate complex. For such an enzyme, in a comparison of Kₘs, a lower Kₘ represents a complex with stronger binding, while a higher Kₘ represents a complex with weaker binding. The ratio k_{cat}/Kₘ, sometimes called the specificity constant, represents the apparent rate constant for combination of substrate with free enzyme. The larger the specificity constant, the more efficient the enzyme is in binding the substrate and converting it to product.

The k_{cat} (also called the turnover number of the enzyme) can be determined if the total enzyme concentration ([E_{T}], i.e., the concentration of active sites) is known, since Vₘₐₓ=k_{cat}[E_{T}]. For situations in which the total enzyme concentration is difficult to measure, the ratio Vₘₐₓ/Kₘ is often used instead as a measure of efficiency. Kₘ and Vₘₐₓ can be determined, for example, from a Lineweaver-Burke plot of 1/V against 1/[S], where the y intercept represents 1/Vₘₐₓ, the x intercept −1/Kₘ, and the slope Kₘ/Vₘₐₓ, or from an Eadie-Hofstee plot of V against V/[S], where the y intercept represents Vₘₐₓ, the x intercept Vₘₐₓ/Kₘ) and the slope −Kₘ. Software packages such as KinetAsyst or Enzfit (Biosoft, Cambridge, UK) can facilitate the determination of kinetic parameters from catalytic rate data.

For enzymes such as polymerases that have multiple substrates, varying the concentration of only one substrate while holding the others constant typically yields normal Michaelis-Menten kinetics. For a more thorough discussion of enzyme kinetics, see, e.g., Berg, Tymoczko, and Stryer (2002) Biochemistry, Fifth Edition, W. H. Freeman; Creighton (1984) Proteins: Structures and Molecular Principles, W. H. Freeman; and Fersht (1985) Enzyme Structure and Mechanism, Second Edition, W. H. Freeman.

In some embodiments, a library of recombinant DNA polymerases can be made and screened/selected for these properties. For example, a plurality of members of the library can be made to include one or more mutation in a region of interest, or even within other genes or regions. Such library is then screened/selected for the desired properties. In general, the library can be tested to identify at least one member comprising a modified activity of interest.

Libraries of polymerases can be either physical or logical in nature. Moreover, any of a wide variety of library formats can be used. For example, polymerases can be fixed to solid surfaces in arrays of proteins. Similarly, liquid phase arrays of polymerases (e.g., in microwell plates) can be constructed for convenient high-throughput fluid manipulations of solutions comprising polymerases. Liquid, emulsion, or gel-phase libraries of cells that express recombinant polymerases can also be constructed, e.g., in microwell plates, or on agar plates. Phage display and/or yeast display libraries of polymerases or polymerase domains (e.g., including an active site region) can be produced. Instructions in making and using libraries can be found throughout the literature, e.g., in Sambrook, Ausubel and Berger, referenced herein.

For the generation of libraries involving fluid transfer to or from microtiter plates, a fluid handling station is optionally used. Several fluid handling stations for performing such transfers are commercially available, including e.g., the Zymate systems from Caliper Life Sciences (Hopkinton, Mass.) and other stations which utilize automatic pipettors. Such systems can optionally be used in conjunction with robotics for plate movement (e.g., the ORCA® robot, Beckman Coulter, Inc. (Fullerton, Calif.), which can be used in a variety of laboratory systems available.

In other embodiments, fluid handling can be performed in microchips, e.g., involving transfer of materials from microwell plates or other wells through microchannels on the chips to destination sites (microchannel regions, wells, chambers or the like).

Commercially available microfluidic systems include those from Hewlett-Packard/Agilent Technologies (e.g., the HP2100 bioanalyzer) and the Caliper High Throughput Screening System. The Caliper High Throughput Screening System provides one example interface between standard microwell library formats and Labchip technologies. The Raindance Technologies droplet-based microfluidics method is another miniaturized system that can be applied to screening enzyme libraries herein. Furthermore, the patent and technical literature includes many examples of microfluidic systems which can interface directly with microwell plates for fluid handling.

Screening/Selecting Methodologies for Identification of Polymerases with Improved Characteristics for Single Molecule Sequencing

One purpose of the invention is to identify polymerases that are better suited for single molecule sequencing. Improvements in polymerase activity can include, e.g., reduced cognate nucleotide disassociation (CND) or “branching” fraction (see below), increased residence time (see below), reduced Kₘ for dye labeled analogs, and difference in incorporation time vs. CND or “branch” time, fidelity, strand displacement, processivity/read length, active fraction, photostability, etc. As explained previously, polymerases comprising one or more of such enhancements will be better suited for use with single molecule sequencing (e.g., in ZMW applications). For example, various polymerases can be selected for one or more of, e.g., a CND or “branching” fraction of <15% or <5% or <1%, a residence time of >40 ms or 100 ms or 50 ms or 20 ms, a rate with four analogues <10 uM of >0.3 Hz or 1 Hz or 5 Hz or 20 Hz, a fidelity of >95% or >97% or >98% or >99%, having strand displacement, a processivity/read length of 100 bp or 1000 bp or 1500 bp or 10 kB, an active fraction of >30% or >80% or >95%, photo stability, etc. Of course, it will be appreciated that such exemplary parameters to optionally be screened/selected for should not necessarily be taken as limiting.

The complexity of polymerase molecules can cause difficulty in identifying independent mutations or combinations of mutations that will produce improvements in characteristics desired for single molecule sequencing. In order to explore the design space of a polymerase in a multifactorial manner, screening/selection processes, as in the instant invention, are designed to allow for the sifting through of many (e.g., >10⁴) polymerase mutations in a simultaneous process. Additionally, many strategies are available for expressing mutated enzymes in a format to allow for selection, screening or enrichment processes to be applied. For example, phage display (or other means of surface display), bead display, or compartmentalized self replication can all optionally be used to express mutant polymerases in a conveniently screenable format. To take advantage of such approaches, the instant invention applies rigorous assay methodologies to ensure the correct enzymatic characteristics can be found. The current invention, when coupled with appropriate techniques that allow for the testing and detection of enzyme functionality, allows the enrichment of populations of enzymes with characteristics of value for single molecule sequencing and other applications.

As explained throughout, the polymerases to be identified through the screens selections herein can be generated in any of a number of ways. For example, directed evolution, a term used to describe various molecular biology techniques that mimic natural selection, can be used to generate polymerases to be screened in the current invention. These directed evolution techniques involve randomly introducing mutations at the genetic level. Such mutation can be followed by screening/selection for the desired characteristics at the protein level using the methods herein. Directed evolution techniques can involve chemical mutagenesis, error-prone PCR, incremental truncation, gene shuffling, etc. The development of directed evolution stemmed from the observation that new protein characteristics can often arise from non-obvious mutations. Alternatively, “rational” design or engineering methods, such as site directed mutagenesis or targeted mutagenesis can also be used with the current invention, combinations of the two, e.g., random mutation of selected polymerase domains, such as the active site, or the nuclease site can also be used. See below. Those of skill in the art will be familiar with a number of mutagenesis techniques and protocols that can be used in conjunction with the screening methods of the current invention.

It will be appreciated that in various embodiments, one or more polymerase (e.g., either randomly or rationally mutated) can be screened/selected through the methods herein and one or more of such screened/selected polymerases (e.g., those showing the best or most desired characteristics) can be either randomly or rationally mutated and then screened/selected as well. Such iterations of mutation and screening/selecting can be repeated any number of times to identify polymerases of interest. Various embodiments of the invention also include wherein beneficial mutations are identified (e.g., during an iteration) and are then added together or recombined into one or more other polymerase variants. Such other variants can then be tested with the methods herein to identify further beneficial combinations, etc.

Again, such iterations can be repeated any number of times.

Again, in addition to directed evolution, rational mutagenesis such as targeted mutagenesis can also be used to generate a range of mutant polymerases to be screened with the current invention. Rational mutagenesis an often be useful in instances where the functionality of interest is well characterized and sufficient information is available to identify the roles of specific amino acids or protein domains in controlling such functionality. Thus, rational mutagenesis can provide successes in generating appropriate modifications in the appropriate settings and can be used to create various polymerases to be screened/selected with the methods herein.

Exemplary Screens/Selections

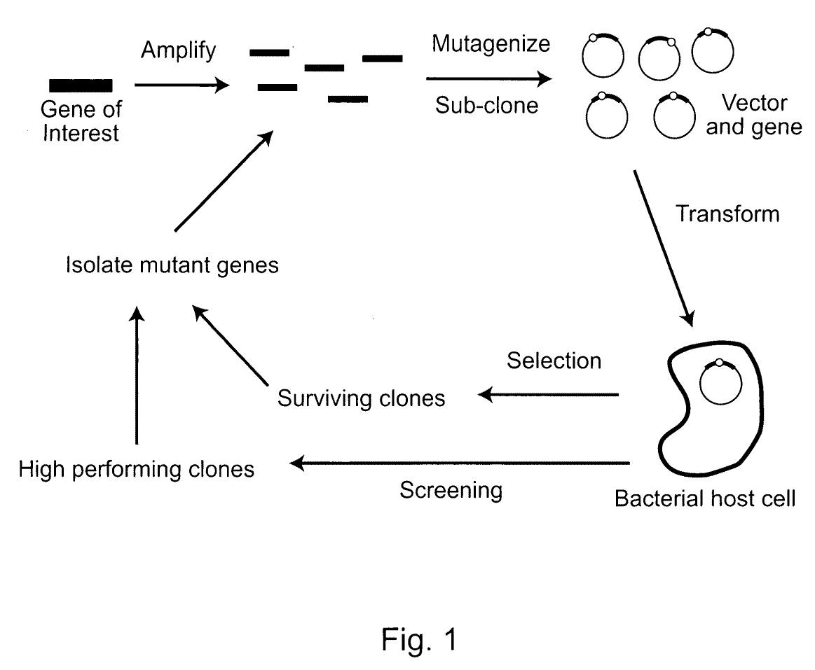

FIG. 1 displays a schematic diagram illustrating an exemplary overview of screening/selecting methods of the invention.

In one example, the methods of the current invention can be used in an overall scheme as follows: First, a range of enzyme mutations are generated (e.g., 10-100 point mutation sites); Second, such mutants are put through one or more screening/selecting tests, e.g., as described herein; Third, the screen/select results are confirmed, e.g., via stop flow and quench flow kinetic measurements; Fourth, a number of regional random mutations are generated (e.g., point mutations); Fifth, such regional mutations are also assayed; Sixth, the results of such screenings/selectings are confirmed via stop flow and quench flow; and Seventh, the identified mutants are used for single molecule sequencing. Stop flow and quench flow measurements are useful for determining enzymatic kinetics for, e.g., fast acting enzymes, and will be familiar to those of skill in the art. Stop flow and quench flow apparatuses are available commercially, e.g., from Kintek, Austin, Tex. It will be appreciated that such exemplary scheme can comprise multiple iterations of screening/selecting and mutagenesis.

Again, in such exemplary situation, initial generation of various point mutations can optionally be designed through rational mutagenesis, e.g., by structural examination of a polymerase such as a phi29 polymerase. Such point mutations can be, e.g., those predominantly designed to improve the kinetics (lower Kₘ) for gamma labeled nucleotide analogs in single molecule sequencing, those designed for use with more than three phosphates in nucleotide analogues, or those designed to create a salt bridge in the closed complex, increasing the residence time or even modifying the CND or “branching” fraction.

In certain embodiments, rather than, or in addition to rational (or even random) point mutations, saturation mutation can be performed to generate mutants to screen with the invention. Thus, regions of the polymerase can be selected and random mutagenesis of these regions by either PCR or oligo based methods can be performed. This procedure can create hundreds of mutants in key regions of the polymerase. Regions that can be subjected to such saturation mutagenesis include, e.g., the fingers, hinge and palm region of the enzyme. Of course, no matter the method of generating mutant, the generation and screening/selecting processes can optionally be iterated numerous times.

Again, it will be appreciated that the screens of the invention can be used with any of a number of different polymerases and their mutations. Thus, different commercially available polymerases and their mutants can be purchased “as is” or can be specially ordered, e.g., from DNA 2.0. Examples of polymerases that can be used in the invention (either as polymerases to be screened or as starting points for creation of mutated/altered polymerases to be screened) include, but are not limited to, Taq polymerases, E. coli DNA Polymerase 1, Klenow fragment, reverse transcriptases, φ29 related polymerases including wild type φ29 polymerase and derivatives of such polymerases such as exonuclease altered forms, an RB69 polymerase phi 15, BS32, M2Y, Nf, G1, Cp-1, PRD1, PZE, SF5, Cp-5, Cp-7, PR4, Pr5, Pr722, L17, T7 DNA polymerase, T5 DNA polymerase, Klenow, N62D, HIV RT, RB 69, KOD, and one phi29 like polymerase (e.g., M2, B103, GA-1, PZA or similar). Other examples of polymerases that can be similarly used are listed throughout.

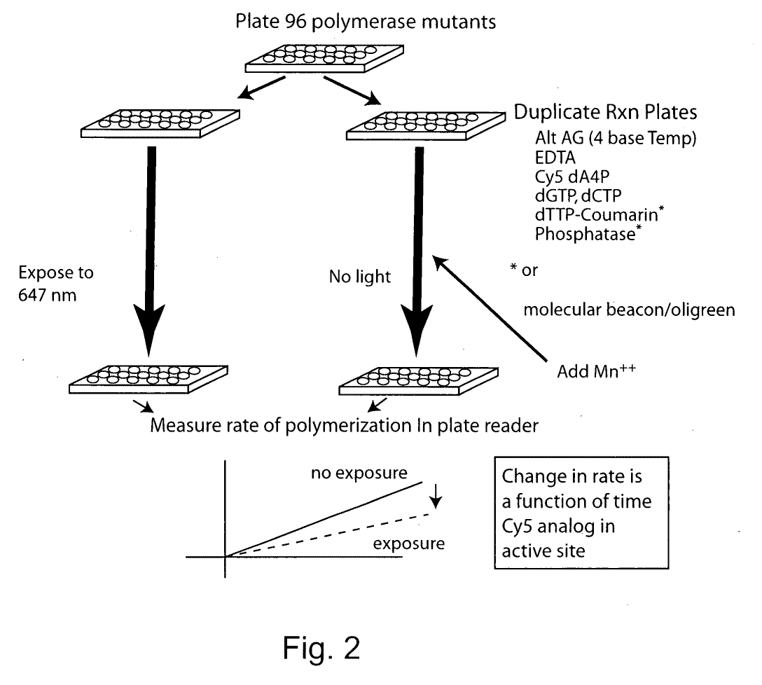

As emphasized throughout, the screens/selections of the invention can comprise a number of different screens/selections, optionally in combination with one another. Thus, for example in some combinations, a group of polymerases (e.g., presented in a library of mutated polymerases) is first screened for activity (i.e., whether the polymerase can synthesize DNA using native dNTPs). The polymerases are then screened for steady state kinetics using, e.g., 1 nucleotide analog and 8 different concentrations to determine Vₘₐₓ and Kₘ. Plate based assays with, e.g., 12 polymerases and one nucleotide analog at 8 concentrations are performed, and the extension rates are measured in a steady state optical readout format to establish estimated Vₘₐₓ and Kₘ. Molecular Beacons, phosphatase dependent fluor, or OliGreen are optionally used in such detection. From the highest concentration of analog in the screen, the rate with, e.g., Mn, or optionally Mg, is measured. Also, residence time and branching fraction are optionally measured for the polymerases. FIG. 2 shows a schematic of exemplary steps involved in a sample residence time screen. The Figure shows a scheme for selecting polymerases based on the time a dye-linked base is present in the active site during the extension reaction. In the embodiment, light exposure is used to inactivate polymerases with high residence times/exposure to the dye molecule. In FIG. 2, the rate of the reaction without photodamage compared to the rate after photodamage is a function of the amount of photodamage caused by the analog in the active site. Such measurement is related to the residence time of the analog and differs between different mutant polymerases.

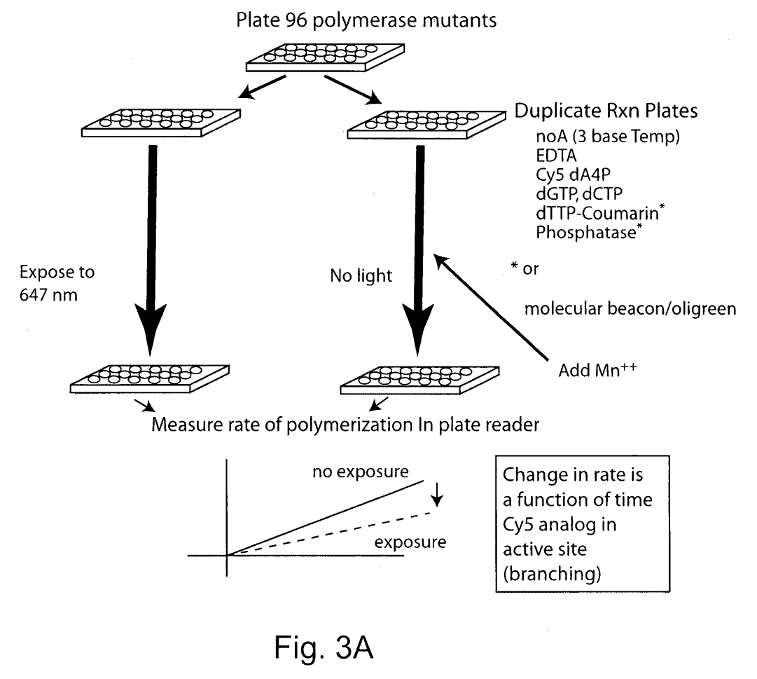

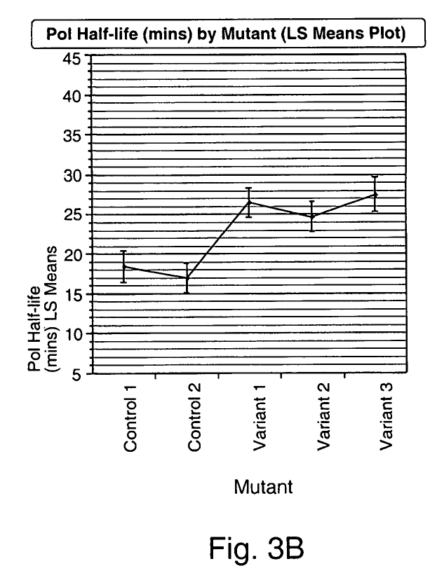

FIG. 3a shows a schematic that illustrates steps involved in a sample screen for CND or “branching” fraction with incorrect nucleotide. In such embodiments, polymerases are selected for based on the number of events of a dye-molecule within the active site —CND configuration. In FIG. 3a, the rate of the reaction without photodamage is compared to the rate after photodamage and is a function of the amount of photodamage caused by the analog in the active site. Such measurement is related to the amount of time that the incorrect nucleotide (analog) spent in the active site, and, thus, a measure of CND fraction. The polymerases are also optionally screened in a native vs. analog competition assay. In such, one tube, or plate reaction, is set up with a primed DNA template, a coumarin-labeled base (native), and three other native nucleotides. The accumulation rate of de-phosphorylated coumarin is measured as endpoint by the addition of SAP and EDTA. The de-phosphorylated coumarin can also be detected as it is produced through monitoring the emission of fluorescence, e.g., in a plate reader. Such real-time reading has the advantage of providing reaction rates. Such measurement is then compared to the size of the DNA fragment generated (e.g., via gel) to produce a comparative rate of the two substrates. The different screens/selections of the invention are optionally applied or reapplied to various polymerases and further mutations or mutation strategies are based on the results of such (e.g., mutation strategies are designed based on the sequence of particular polymerases, e.g., those with desired traits). FIG. 3b compares the half-life of two unscreened/unselected polymerases against three polymerases that have been screened/selected for photostability. See Example 1 below.

Fluor-Dependent Photostability

In some embodiments, the current invention comprises a screen for fluor-dependant photostability. In particular single molecule sequencing reactions, such as those utilizing ZMWs, the polymerase is exposed to light in various wavelengths and in the presence and/or absence of particular fluorophores. Thus, it is desirable to have polymerases that display photostability. In exemplary screens for photostability in the presence of fluorophores, the mutant library (which can be created, e.g., as described herein) is exposed to a fluorescent chemistry (either independent or covalently linked to other molecules). Exposure to a light excites the fluor molecule and allows for photodamage activity to occur (if it does). After exposure, reagents that will support a polymerase extension product are added (in the presence of an appropriate template with primer, nucleotides, etc.). Detection of an extension reaction product can be monitored by incorporation of a fluorescently labeled nucleotide at an incorporation site distant from the initiation point using a nucleotide linked to a fluorescent molecule in a configuration where the fluorescent molecule remains incorporated into the extended strand. Thus, the presence of an incorporated fluorescent base (monitored, e.g., by FACS sorting or through use of, e.g., imaging instruments such as Typhoon from Molecular Dynamics,) is an indicator of survival after photodamaging conditions have been applied. This screen, thus, identifies polymerases that are resistant to photodamage in the presence of a fluorophore. In various embodiments, the fluorophore is physically connected or tethered to an oligonucleotide that can hybridize to a nucleic acid template upon which the polymerase acts. Such tethering helps to keep the fluorophore in the correct location (e.g., prevents the fluorophore from floating away). It will be appreciated that a wide range of fluorophores of various types can be used in such embodiments. Also, in various embodiments, the fluorophore will be located at or near the 3′ end of the oligonucleotide such that it will be in close proximity to the binding pocket of the polymerase.

Fluor-independent Photostability

In another embodiment, the invention comprises a screen for fluor-independent photostability. Such screen is similar to that for the fluor-dependent screen above, but without the inclusion of a fluorescent chemistry prior to the light exposure. Different wavelengths of light may be tested in either of such configurations.

Residence Time

In yet other embodiments, the invention comprises a screen for residence time. In such screens, using an expressed mutant library, non-functional mutants can first be eliminated by performing a “non-limiting” extension reaction whereby all mutants capable of extending are selected for, e.g., by detecting incorporation of fluorescent nucleotides. This selected pool of active mutants can then be further tested for rate of primer extension in subsequent screen rounds by performing “limiting” extension reactions where the limitation is time, and the metric is the ability of the polymerase to result in incorporation of a fluorescent base at a set distance (in units of nucleotides) from the initiation point. Two differing fluorescent nucleotides can be used—one to indicate the presence of a “near” incorporation, with a further nucleotide being used to indicate the incorporation of a “far” incorporation. A successful incorporation into only the near but not the far site can be indicative of mutants that are slow to incorporate. Such slow incorporation can be due to, e.g., extension in the residence time. Conditions to exacerbate a slow incorporation rate (such as lower temperature, suboptimal pH or buffer conditions) can be used to facilitate the screening process. Various embodiments can comprise identification of polymerases displaying increased residence time, while other embodiments can comprise identification of polymerases having decreased residence time.

Affinity

In other embodiments, the invention comprises a screen for altered affinity (Kₘ). Using an expressed mutant library, non-functional mutants can be eliminated by performing, in the presence of an appropriately designed template:primer, a “non-limiting” extension reaction whereby all mutants capable of extending are selected for, e.g., by incorporation of fluorescent nucleotides. Taking the pool of active mutants thereby selected, a subsequent screening round further tests for affinity of binding by performing extension reactions in the presence of “limiting” amounts of nucleotide bases. High affinity is indicated by successful extension under the condition of low analog concentration. Selection is made by observation of the incorporation of a fluorescent nucleotide distantly placed from the 3′ OH initiation point of the reaction.

Cation Selection

The invention also comprises embodiments in which polymerases are screened based on cation selection. In such embodiments, polymerase mutants (e.g., in libraries, etc.) are provided with different divalent cations such as Mn²⁺, Ca²⁺, Co²⁺, Ca²⁺, Sr²⁺, Ba²⁺ or the like and/or any combinations of such or similar cations, and their ability to incorporate a fluorescently labeled nucleotide (or other marker) is then monitored. In yet other embodiments, a slow incorporation rate in the presence of such different divalent cations can be screened for by combining the substitution of divalent cations with time selection under conditions where extension rates are slowed due to pH or temperature variations. Here too, as optionally with any of the screens/selections herein, the mutagens screened in any iteration can first be selected for functional mutants. See above.

CND (“Branching”)

In yet other embodiments, the invention comprises a selection for cognate nucleotide disassociation (“branching”). CND is the rate of dissociation of a nucleotide or nucleotide analogue from the polymerase active site without incorporation of the nucleotide or nucleotide analogue, where the nucleotide or nucleotide analogue, if it were incorporated, would correctly base-pair with a complementary nucleotide or nucleotide analogue in the template. During a polymerase kinetic cycle, sampling of each of four possible nucleotides (or analogues) occurs until a correct Watson-Crick pairing is generated (see, e.g., Hanzel WO 2007/076057 POLYMERASES FOR NUCLEOTIDE ANALOGUE INCORPORATION for a description of the kinetic cycle of a polymerase). However, chemical linkages between a sampled nucleotide and a 3′OH group of a preceding base can fail to occur for a correctly paired nucleotide, due to release of the correctly paired base from the active site. Such failures to physically incorporate the correct nucleotide can result in sequence read errors in single-molecule sequencing by incorporating methods. The polymerase kinetic cycle is repeated for the same site, eventually resulting in actual physical incorporation of the correct nucleotide at the site. However, where both the failed incorporation and the actual incorporation of the nucleotides are read by the system as incorporation events, sequences deciphered during single molecule sequencing (SMS) for the incorporation site have an incorrect “insertion” relative to the correct sequence. This cognate nucleotide dissociation can be termed “branching” because it leads to a “branch” in the sequence (a site where two identical molecules will be read as having different sequences) and can ultimately generate high error rates during single molecule sequencing.

CND screening embodiments are similar to others herein, but the library of mutant polymerases is provided with a divalent cation that allows for a cognate base to correctly Watson—Crick pair. However, where the reaction conditions preclude extension, the active complex can be created in a static non-extending configuration. Subsequent saturation with a dideoxy-nucleotide or another non-hydrolyzable analog bearing a fluorescent signal, in concert with a divalent cation and extendable base chemistries that allow extension by multiple bases, will result in sites that are open at the time of this addition being terminated by the binding of the non-hydrolyzable analog. Sites that contain the cognate, hydrolyzable base proceed with extension and generate an extension product that can be detected by the incorporation of a differently labeled fluorescently-labeled nucleotide at a downstream extension site. In some embodiments, the CND fraction can be measured by “loading” a polymerase active site with a cognate-matching nucleotide analog that can bind in the +1 and +2 positions. In the absence of divalent cation this nucleotide cannot be incorporated into the DNA strand, so will pair with the template nucleotide at the +1 position but be released at some frequency specific for that analog/polymerase combination, e.g., the branching rate. This ‘loading’ reaction is then followed by a ‘chase’ reaction consisting of a divalent cation that supports extension, e.g., Mn2+), and a terminating-type nucleotide analog, e.g., a dideoxynucleotide, comprising the same base as the cognate-matching analog in the loading step.

By choice of polymerases that produce non-extended/extended nucleotide products, two pools can therefore be enriched for: those that have increased CND, i.e., “branch” frequently and those that have decreased CND, i.e., “branch” less frequently. Iterative selections of this nature in the presence of different concentrations of analog can be performed to determine CND rates relative to affinity. Illustration of the result of screening polymerases for the characteristic of decreased cognate nucleotide dissociation can be seen in Example 2.

In some embodiments, the “branching fraction” can be determined by the proportion of cognate nucleotide (or nucleotide analog) dissociation events from the polymerase active site to the total number of events, e.g., the sum of the incorporation events and dissociation events.

Fidelity

In yet other embodiments, the invention comprises screens for fidelity. In such embodiments, a library of mutant polymerases is provided with a circular template:primer which will allow the production of long (>1 Kb) products containing only 3 bases (e.g., A, T, C). Extension reactions in the presence of 3 bases where one base differs (e.g., A, T, G) results in extension only in the case where a mismatch has occurred. Detection of the extension product can be made by incorporation of the mismatched nucleotide containing a fluorescently labeled base. Those mutants that do not generate a fluorescent product are identified as having greater fidelity. Greater stringency of this screen can be imposed by biasing (increasing) the concentrations of the mismatch base relative to the correct incorporations or by altering reaction conditions such as choice of divalent cation, buffer, pH or temperature, to force greater mismatch rates to occur.

Exonuclease Activity

In yet other embodiments, the invention comprises a screen for exonuclease activity. Such embodiments screen for polymerases having reduced 3′ to 5′ exonuclease activity. Such screens are useful because nuclease is typically undesirable in most DNA sequencing schemes and many DNA polymerases also have nuclease activity. In such embodiments, polymerase mutants are screened against primer:template complexes in which the primer has a detectable signal near the 3′ end. Therefore, if the polymerase degrades the labeled nucleotide via 3′-5′ exonuclease activity, the signal will be lost. The signal can be, e.g., fluorescence, affinity (e.g., biotin), radioactivity, e.g., ³²P, ³⁵S, etc. The label can optionally be at the 3′ end or can be at the −1, −2, etc., position to test different extents of exonuclease activity. One advantage of embodiments having a screen for degradation of the maker is that polymerases lacking nuclease activity retain the signal, which makes screening easier. However, in other embodiments, the signal can be “off” (quenched) until exonuclease activity turns the signal “on.” In such instances, an exonuclease-deficient polymerase would not activate the signal. An example is a 2-amino-purine nucleotide that has a lower signal in the context of duplex DNA than in the context of being at the 3′ end. Nuclease activity exposes the 2-AP and produces a signal. Advantages to such embodiment are that a gain of a signal is often preferential in order to measure activity and that the nuclease would be acting on native nucleotides and not dye-labeled or other analogs.

It will be appreciated that in the various screening/selecting embodiments herein that multiple detection methodologies can be employed. Thus, while particular illustrations herein may rely on the incorporation of a fluorescent molecule covalently linked to the nucleotide of choice to determine the length or presence of a DNA strand produced (e.g., with the labeled nucleotide being incorporated at a site distal to the initiation point), other fluorescent detection strategies can be employed. Such strategies can be, but are not limited to, molecular beacon or OliGreen (e.g., selection for amount of mass of DNA produced). Alternatively, non-fluorescent detection procedures, such as binding enrichment via biotin or other type of affinity tag can also be used.

Also, it will be appreciated that the various screens/selections herein can be combined in various ways. For example, some embodiments of the invention can comprise multiple iterations of screenings (with optional mutation rounds between the screenings) wherein each screening is for a different characteristic, e.g., increased CND in iteration one, increased processivity in iteration 2, etc. In other embodiments, more than one characteristic can be screened for simultaneously in the same iteration, e.g., increased CND and increased processivity screened for in the same iteration.

### Nucleic Acid Oligonucleotide Probe Hybridization

In yet other embodiments of the invention, detection of the location of specific sequences within nucleic acids generated from polymerization reactions can be achieved in a “non-sequencing” mode by hybridizing a complementary oligonucleotide containing a fluorescent molecule (preferably, but not necessarily, quenched by a proximal quenching chemistry) to a specific generated sequence. This can be used to generate measure lengths of DNA produced per unit time, absolute lengths of DNA produced and identification of the presence of specific sequences generated during polymerization reactions.

Fluorescently labeled and quenched oligonucleotide probes provide a low signal to noise background until the fluor and quencher molecule are separated spatially. This spatial separation can be achieved via hybridization of an intervening DNA sequence to a highly homologous product strand, or by cleavage of the quenching molecule via enzymatic digestion. These technologies are routinely used in bulk assays for genomic applications. However, embodiments of the invention apply these approaches at the single-molecule level for identification of the presence of specific sequences as they are produced, e.g., in a ZMW from extension by a functional polymerase. DNA hybridization strategies in such embodiments can include various designs including molecular beacons (see, e.g., Nilsson, et al., Nuc. Acids Res., 2002, 30(14)e66), adjacent probes, 5′ nuclease probes, and light-up probes. It will be appreciated that such oligonucleotide hybridization con optionally be used with the various screens/selections herein to monitor enzyme activity, etc.

### Tracking in Single Molecule Enzyme Screening Methods

In yet additional embodiments, the invention comprises methods of performing high throughput screening of enzyme mutations. In traditional methodology, clones of cross-over or point mutations have to be screened using standard microwell plates and microgram quantities of protein. While this method is adequate when screening hundreds of clones, it can become a bottle-neck as the number of clones reaches into the thousands or tens of thousands. Thus, some embodiments herein address this problem by comprising a method that allows the simultaneous screening of thousands or tens of thousands of clones at once using sub-microgram quantities of protein. In addition, such embodiments provide a way to uniquely tag each clone of interest for subsequent identification and tracking.

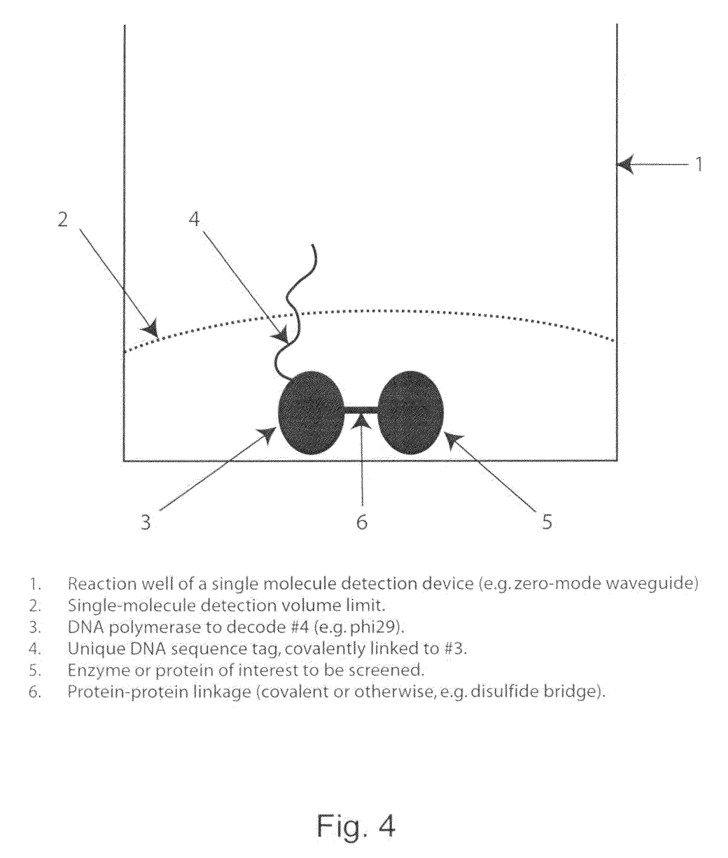

The unique DNA identification tag used in such embodiments can be altered in length to represent the desired number of unique tags. The composition of the ID tag can be altered to use modified bases (e.g., PNAs) as long as they can be processed by the decoding polymerase. The decoding polymerase also can be altered to have different enzymatic characteristics. For example, if the enzyme being screened is itself a polymerase (such as in other embodiments herein), the activity of the “decoding” polymerase can be altered to be temperature, salt, pH or ligand sensitive, allowing it to be activated for decoding only after the polymerase activity has been determined (e.g., as seen in the above screening examples). Furthermore, in some embodiments, the polymerase can be manipulated to determine (or “read”) the sequence of its own DNA tag. Doing so may fulfill two roles simultaneously, namely determination of whether a polymerase is active in a given condition, and determination of the identification of the DNA tag. Finally, the linkage between the decoding polymerase and the protein or enzyme being screened can be modified to include covalent linkages, hydrophobic linkages, or even hybridization linkages (using complementary DNA strands). Of course, it will be appreciated, that while such DNA identification tags can optionally be utilized with the other polymerase screening embodiments herein, enzymes other than polymerases can also be used. FIG. 4 shows a schematic illustrating an exemplary ID tag and polymerase.

Selection Methods for In vitro Molecular Evolution of DNA Polymerase Function by Phage Display

As illustrated throughout, in vitro evolution of DNA polymerase with novel functions is a powerful strategy to custom generate an enzyme that is optimized in performance for single molecule sequencing technology. Creating the correct selection criteria and improving the screening process ensures that the “correct” enzyme is identified (i.e., one that is most suitable for its intended purpose). Thus, in some embodiments herein, phage display methods to evolve DNA polymerases with novel functions can be used. Phage display has been known and widely applied in the biological sciences and biotechnology. See, e.g., Xia, et al. (2002), PNAS 99:6597-6602; and U.S. Pat. Nos. 5,223,409; 5,403,484; 5,4571,698; and 5,766,905; and the references cited therein. It can be important to screen and select for function such as strand displacement DNA synthesis and high processivity.

In phage display evolution, desired polymerase functions such as strand displacement DNA synthesis and/or processivity are optionally implemented as described herein. For DNA strand displacement synthesis, a hairpin DNA template is used that contains either an unnatural nucleobase which the polymerase is not able to synthesize through, or only 3 of the 4 DNA bases in the stem of the hairpin (with the fourth base in the loop). Thus only 3 of the 4 dNTPs are utilized for extension selection. In this strategy, a polymerase mutant from the phase display can only be selected if it is able to strand displace through the stem region of the hairpin and then stop at the unnatural or missing dNTP site, respectively. This exposes the displaced stem, which can be hybridized to an oligonucleotide that is biotinylated or on a solid support for isolation of the desired mutants.

For identification of high processivity, the polymerase mutant can optionally be preincubated with its template in the absence of dNTPs or Mg ions, and then the DNA synthesis reaction initiated with such (and including heparin, or a large excess of unlabeled template, as a processivity trap). Heparin, a DNA mimic, is included in processivity assays to ensure all products are generated via processive reaction. Non-processive reactions are precluded by binding of released (non-processive) polymerases to the heparin “trap.” Heparin is used to titrate out DNA-binding molecules such as DNA polymerases and thus prevent them from re-binding to DNA. Free DNA, e.g., primer:template partial DNA duplex, can likewise be used to “trap” non-processive enzymes. Any non-processive synthesis thus results in polymerase binding to the processivity trap heparin, and the biotin-dUTP incorporation site on the template not being reached. Non-processive mutants are thus excluded from the selection.

In such embodiments, it can be desirable to use all four analogs replacing native dNTPs during the screen. Such thus avoids having to cycle the biotinylated base linked dNTP in successive rounds. Towards that end, the polymerase can be allowed to synthesize the entire strand, which results in a blunt end which can then be selected for by blunt end ligation of a biotinylated or otherwise selectable entity. Alternatively, the template can contain an unnatural base, which results in a stop of DNA synthesis. The remaining ssDNA template can then be used to hybridize to the selectable probe, such as a biotinylated oligonucleotide.

Yeast surface display can be used as an alternative to phage display. In yeast display many more copies of the protein are displayed per particle (tens of thousands per yeast cell vs. ˜1 per phage particle). Additionally, yeast are bigger and easier to sort by FACS than typical phage. See, for example, Chao, et al. (2006), Nature Protocols 1:755-768.

### Affinity Tags And Other Optional Polymerase Features

The DNA polymerases screened/selected herein optionally include additional features exogenous or heterologous to the polymerase. For example, the polymerases can optionally include one or more exogenous affinity tags, e.g., purification or substrate binding tags, such as a 6-His tag sequence, a GST tag, an HA tag sequence, a plurality of 6-His tag sequences, a plurality of GST tags, a plurality of HA tag sequences, a SNAP-tag, or the like. These and other features useful in the context of binding a polymerase to a surface are optionally included, e.g., to orient and/or protect the polymerase active site when the polymerase is bound to a surface. Other useful features include recombinant dimer domains of the enzyme, and, e.g., large extraneous polypeptide domains coupled to the polymerase distal to the active site. For example, for q)₂₉, the active site is in the C terminal region of the protein, and added surface binding elements (extra domains, His tags, etc.) are typically located in the N-terminal region to avoid interfering with the active site when the polymerase is coupled to a surface.

In general, surface binding elements and purification tags that can be added to the polymerase (recombinantly or, e.g., chemically) include, e.g., polyhistidine tags, 6-His tags, biotin, avidin, GST sequences, biotin-ligase-recognition sequences, S tags, SNAP-tags, enterokinase sites, thrombin sites, antibodies or antibody domains, antibody fragments, antigens, receptors, receptor domains, receptor fragments, ligands, dyes, acceptors, quenchers, or combinations thereof.

Multiple surface binding domains can be added to orient the polypeptide relative to a surface and/or to increase binding of the polymerase to a surface or functionality via the surface. By binding a surface at two or more sites, through two or more separate tags, the polymerase can be held in a relatively fixed orientation with respect to the surface. Additional details on fixing a polymerase to a surface are found in U.S. Patent Application 60/753,446 “PROTEIN ENGINEERING STRATEGIES TO OPTIMIZE ACTIVITY OF SURFACE ATTACHED PROTEINS” by Hanzel, et al. and U.S. Patent Application 60/753,515 “ACTIVE SURFACE COUPLED POLYMERASES” by Hanzel, et al., both filed Dec. 22, 2005 and incorporated herein by reference for all purposes, and in U.S. patent application Ser. No. 11/645,135 “PROTEIN ENGINEERING STRATEGIES TO OPTIMIZE ACTIVITY OF SURFACE ATTACHED PROTEINS” by Hanzel, et al., and U.S. patent application Ser. No. 11/645,125 “ACTIVE SURFACE COUPLED POLYMERASES” by Hanzel, et al. both filed on Dec. 21, 2006 and both incorporated herein by reference for all purposes.

### Making and Isolating Recombinant Polymerases

Generally, nucleic acids encoding a polymerase to be screened/selected by the invention can be made by cloning, recombination, in vitro synthesis, in vitro amplification and/or other available methods. A variety of recombinant methods can be used for expressing an expression vector that encodes a polymerase, e.g., a mutant polymerase, to be screened/selected herein. Recombinant methods for making nucleic acids, expression and isolation of expressed products are described, e.g., in Berger and Kimmel, Guide to Molecular Cloning Techniques, Methods in Enzymology volume 152 Academic Press, Inc., San Diego, Calif. (Berger); Sambrook, et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel, et al., eds., Current Protocols, a joint venture between Greene Publishing Associates, Inc. and John Wiley & Sons, Inc; Kaufman, et al. (2003) Handbook of Molecular and Cellular Methods in Biology and Medicine Second Edition Ceske (ed.) CRC Press (Kaufman); and The Nucleic Acid Protocols Handbook Ralph Rapley (ed.) (2000) Cold Spring Harbor, Humana Press Inc (Rapley).

Additionally, a wide range of kits are commercially available for plasmid purification or purification of other relevant nucleic acids from cells, (e.g., EasyPrep™, FlexiPrep™, both from Pharmacia Biotech; StrataClean™, from Stratagene; and, QIAprep™ from Qiagen). Any isolated and/or purified nucleic acid can be further manipulated to produce other nucleic acids, used to transfect cells, incorporated into related vectors to infect organisms for expression, and the like. Typical cloning vectors contain transcription and translation terminators, transcription and translation initiation sequences, and promoters useful for regulation of the expression of a particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Thus, vectors can be suitable for replication and integration in prokaryotes, eukaryotes, or both. See, Giliman & Smith, Gene 8:81 (1979); Roberts, et al., Nature, 328:731 (1987); Schneider, B., et al., Protein Expr. Purif. 6435:10 (1995); Ausubel, Sambrook, Berger (above). A catalogue of Bacteria and Bacteriophages useful for cloning is provided, e.g., by the ATCC, e.g., The ATCC Catalogue of Bacteria and Bacteriophage published yearly by the ATCC. Additional basic procedures for sequencing, cloning and other aspects of molecular biology and underlying theoretical considerations are also found in Watson, et al. (1992) Recombinant DNA Second Edition, Scientific American Books, NY.

Other useful references, e.g. for cell isolation and culture (e.g., for subsequent nucleic acid isolation of mutant polymerases) include Freshney (1994) Culture of Animal Cells, a Manual of Basic Technique, third edition, Wiley-Liss, New York and the references cited therein; Payne, et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg N.Y.) and Atlas and Parks (eds.) The Handbook of Microbiological Media (1993) CRC Press, Boca Raton, Fla. Furthermore, essentially any nucleic acid (e.g., a nucleic acid of a polymerase to be screened) can be custom or standard ordered from any of a variety of commercial sources, such as Operon Technologies Inc. (Alameda, Calif.).

A variety of protein isolation and detection methods are known and can be used to isolate polymerases, e.g., from recombinant cultures of cells expressing mutant polymerases to be screened/selected herein. Also, a variety of protein isolation and detection methods are well known in the art, including, e.g., those set forth in Deutscher, Methods in Enzymology Vol. 182: Guide to Protein Purification, Academic Press, Inc. N.Y. (1990); Sandana (1997) Bioseparation of Proteins, Academic Press, Inc.; Bollag, et al. (1996) Protein Methods, 2^{nd} Edition Wiley-Liss, NY; Walker (1996) The Protein Protocols Handbook Humana Press, NJ; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

### Mutating Polymerases

Various types of mutagenesis, e.g., as mentioned above, can optionally be used to create polymerases to be screened/selected by the current invention. In general, any available mutagenesis procedure can be used for making such mutants. After such mutagenesis, the invention comprises screening/selection of the mutant polypeptides for one or more activity of interest (e.g., any of those described above such as improved Kₘ, Vₘₐₓ, k_{cat} etc.). Procedures that can be used include, but are not limited to: site-directed point mutagenesis, random point mutagenesis, in vitro or in vivo homologous recombination (DNA shuffling), mutagenesis using uracil containing templates, oligonucleotide-directed mutagenesis, phosphorothioate-modified DNA mutagenesis, mutagenesis using gapped duplex DNA, mutagenesis by overlap extension PCR, point mismatch repair, mutagenesis using repair-deficient host strains, restriction-selection and restriction-purification, deletion mutagenesis, mutagenesis by total gene synthesis, degenerate PCR, double-strand break repair, and many others known to persons of skill in the art.

Optionally, mutagenesis can be guided by known information from a naturally occurring polymerase molecule, or from a known altered or mutated polymerase (e.g., an existing mutant polymerase that displays a desired characteristic). Such information can include, e.g., sequence, sequence comparisons, physical properties, crystal structure and/or the like. Thus, in particular uses, modification can be essentially random, or can be directed/designed based on known parameters.

The polymerase mutational strategies noted herein can be combined with other available mutations and mutational strategies to confer additional putative improvements in, e.g., nucleotide analog specificity, enzyme processivity, etc. For example, the mutational strategies herein can be combined with those taught in, e.g., WO 2007/076057 POLYMERASES FOR NUCLEOTIDE ANALOGUE INCORPORATION by Hanzel, and PCT/US2007/022459 POLYMERASE ENZYMES AND REAGENTS FOR ENHANCED NUCLEIC ACID SEQUENCING by Rank. Such combinations of strategies can be used to try to impart several simultaneous improvements to a polymerase (e.g., decreased CND fraction formation, improved specificity, improved processivity, improved retention time, etc.). Polymerases created through such combined strategies can be screened/selected through the methods herein.

Additional information on mutation formats an be found in: Sambrook, et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel, et al., eds., Current Protocols, a joint venture between Greene Publishing Associates, Inc. and John Wiley & Sons, Inc., (supplemented through 2006) (“Ausubel”)) and PCR Protocols A Guide to Methods and Applications (Innis, et al. eds) Academic Press Inc. San Diego, Calif. (1990) (Innis). The following publications and references provide additional detail on mutation formats that can be used to generate polymerases to be screened/selected through the methods herein: Arnold, “Protein engineering for unusual environments,” Current Opinion in Biotechnology 4:450-455 (1993); Bass, et al., “Mutant Trp repressors with new DNA-binding specificities,” Science 242:240-245 (1988); Botstein & Shortle, “Strategies and applications of in vitro mutagenesis,” Science 229:1193-1201 (1985); Carter, et al., “Improved oligonucleotide site-directed mutagenesis using M13 vectors,” Nucl. Acids Res. 13: 4431-4443 (1985); Carter, “Site-directed mutagenesis,” Biochem. J. 237:1-7 (1986); Carter, “Improved oligonucleotide-directed mutagenesis using M13 vectors,” Methods in Enzymol. 154: 382-403 (1987); Dale, et al., “Oligonucleotide-directed random mutagenesis using the phosphorothioate method,” Methods Mol. Biol. 57:369-374 (1996); Eghtedarzadeh & Henikoff, “Use of oligonucleotides to generate large deletions,” Nucl. Acids Res. 14: 5115 (1986); Fritz, et al., “Oligonucleotide-directed construction of mutations: a gapped duplex DNA procedure without enzymatic reactions in vitro,” Nucl. Acids Res. 16: 6987-6999 (1988); Ho, et al., “Site-directed mutagenesis by overlap extension using the polymerase chain reaction,” Gene 77: 51-59 (1989); Higuchi, et al., “A general method of an in vitro preparation and specific mutagenesis of DNA fragments: study of protein and DNA interactions,” Nucl. Acids Res. 16:7351-7367, (1988); Grundstrom, et al., “Oligonucleotide-directed mutagenesis by microscale ‘shot-gun’ gene synthesis,” Nucl. Acids Res. 13: 3305-3316 (1985); Kunkel, “The efficiency of oligonucleotide directed mutagenesis,” in Nucleic Acids & Molecular Biology (Eckstein, F. and Lilley, D. M. J. eds., Springer Verlag, Berlin)) (1987); Kunkel, “Rapid and efficient site-specific mutagenesis without phenotypic selection,” Proc. Natl. Acad. Sci. USA 82:488-492 (1985); Kunkel, et al., “Rapid and efficient site-specific mutagenesis without phenotypic selection,” Methods in Enzymol. 154, 367-382 (1987); Kramer, et al., “The gapped duplex DNA approach to oligonucleotide-directed mutation construction,” Nucl. Acids Res. 12: 9441-9456 (1984); Kramer & Fritz “Oligonucleotide-directed construction of mutations via gapped duplex DNA,” Methods in Enzymol. 154:350-367 (1987); Kramer, et al., “Point Mismatch Repair, “Cell 38:879-887 (1984); Kramer, et al., “Improved enzymatic in vitro reactions in the gapped duplex DNA approach to oligonucleotide-directed construction of mutations, “Nucl. Acids Res. 16: 7207 (1988); Ling, et al., “Approaches to DNA mutagenesis: an overview,” Anal. Biochem. 254(2): 157-178 (1997); Lorimer and Pastan Nucleic Acids Res. 23, 3067-8 (1995); Mandecki, “Oligonucleotide-directed double-strand break repair in plasmids of Escherichia coli: a method for site-specific mutagenesis,” Proc. Natl. Acad. Sci. USA, 83:7177-7181 (1986); Nakamaye & Eckstein, “Inhibition of restriction endonuclease Nci I cleavage by phosphorothioate groups and its application to oligonucleotide-directed mutagenesis,” Nucl. Acids Res. 14:9679-9698 (1986); Nambiar, et al., “Total synthesis and cloning of a gene coding for the ribonuclease S protein,” Science 223: 1299-1301 (1984); Sakamar and Khorana, “Total synthesis and expression of a gene for the a-subunit of bovine rod outer segment guanine nucleotide-binding protein (transducin),” Nucl. Acids Res. 14: 6361-6372 (1988); Sayers, et al., “Y-T Exonucleases in phosphorothioate-based oligonucleotide-directed mutagenesis,” Nucl. Acids Res. 16:791-802 (1988); Sayers, et al., “Strand specific cleavage of phosphorothioate-containing DNA by reaction with restriction endonucleases in the presence of ethidium bromide,” (1988) Nucl. Acids Res. 16: 803-814; Sieber, et al., Nature Biotechnology, 19:456-460 (2001); Smith, “In vitro mutagenesis,” Ann. Rev. Genet. 19:423-462 (1985); Methods in Enzymol. 100: 468-500 (1983); Methods in Enzymol. 154: 329-350 (1987); Stemmer, Nature 370, 389-91 (1994); Taylor, et al., “The use of phosphorothioate-modified DNA in restriction enzyme reactions to prepare nicked DNA,” Nucl. Acids Res. 13: 8749-8764 (1985); Taylor, et al., “The rapid generation of oligonucleotide-directed mutations at high frequency using phosphorothioate-modified DNA,” Nucl. Acids Res. 13: 8765-8787 (1985); Wells, et al., “Importance of hydrogen-bond formation in stabilizing the transition state of subtilisin,” Phil. Trans. R. Soc. Lond. A 317: 415-423 (1986); Wells, et al., “Cassette mutagenesis: an efficient method for generation of multiple mutations at defined sites,” Gene 34:315-323 (1985); Zoller & Smith, “Oligonucleotide-directed mutagenesis using M13-derived vectors: an efficient and general procedure for the production of point mutations in any DNA fragment,” Nucleic Acids Res. 10:6487-6500 (1982); Zoller & Smith, “Oligonucleotide-directed mutagenesis of DNA fragments cloned into M13 vectors,” Methods in Enzymol. 100:468-500 (1983); and Zoller & Smith, “Oligonucleotide-directed mutagenesis: a simple method using two oligonucleotide primers and a single-stranded DNA template,” Methods in Enzymol. 154:329-350 (1987). Additional details on many of the above methods can be found in Methods in Enzymology Volume 154, which also describes useful controls for trouble-shooting problems with various mutagenesis methods.

### Specific Modifications to DNA Polymerases to Produce Desired Characteristics

Structure-Based Design of Recombinant Polymerases

Structural data for a polymerase can be used to conveniently identify amino acid residues as candidates for mutagenesis to create recombinant polymerases putatively having modified characteristics which are then screened/selected through use of the instant invention. For example, analysis of the three-dimensional structure of a polymerase can identify residues that can be mutated to introduce a desired feature.

The three-dimensional structures of a large number of DNA polymerases have been determined by x-ray crystallography and nuclear magnetic resonance (NMR) spectroscopy, including the structures of polymerases with bound templates, nucleotides, and/or nucleotide analogues and the like. Many such structures are freely available for download from the Protein Data Bank, at (www.rcsb.org/pdb). Structures, along with domain and homology information, are also freely available for search and download from the National Center for Biotechnology Information's Molecular Modeling DataBase, at www.ncbi.nlm.nih.gov/Structure/MMDB/mmdb.shtml. The structures of additional polymerases can be modeled, for example, based on homology of the polymerases with polymerases whose structures have already been determined. Alternatively, the structure of a given polymerase, can be determined.

Techniques for crystal structure determination are well known. See, for example, McPherson (1999) Crystallization of Biological Macromolecules Cold Spring Harbor Laboratory; Bergfors (1999) Protein Crystallization International University Line; Mullin (1993) Crystallization Butterwoth-Heinemann; Stout and Jensen (1989) X-ray structure determination: a practical guide, 2nd Edition Wiley Publishers, New York; Ladd and Palmer (1993) Structure determination by X-ray crystallography, 3rd Edition Plenum Press, New York; Blundell and Johnson (1976) Protein Crystallography Academic Press, New York; Glusker and Trueblood (1985) Crystal structure analysis: A primer, 2nd Ed. Oxford University Press, NewYork; International Tables for Crystallography, Vol. F. Crystallography of Biological Macromolecules; McPherson (2002) Introduction to Macromolecular Crystallography Wiley-Liss; McRee and David (1999) Practical Protein Crystallography, Second Edition Academic Press; Drenth (1999) Principles of Protein X-Ray Crystallography (Springer Advanced Texts in Chemistry) Springer-Verlag; Fanchon and Hendrickson (1991) Chapter 15 of Crystallographic Computing, Volume 5 IUCr/Oxford University Press; Murthy (1996) Chapter 5 of Crystallographic Methods and Protocols Humana Press; Dauter, et al. (2000) “Novel approach to phasing proteins: derivatization by short cryo-soaking with halides” Acta Cryst. D 56:232-237; Dauter (2002) “New approaches to high-throughput phasing” Curr. Opin. Structural Biol. 12:674-678; Chen, et al. (1991) “Crystal structure of a bovine neurophysin-II dipeptide complex at 2.8 Å determined from the single-wavelength anomalous scattering signal of an incorporated iodine atom” Proc. Natl. Acad. Sci. USA, 88:4240-4244; and Gavira, et al. (2002) “Ab initio crystallographic structure determination of insulin from protein to electron density without crystal handling” Acta Cryst.D 58:1147-1154.

In addition, a variety of programs to facilitate data collection, phase determination, model building and refinement, and the like are publicly available. Examples include, but are not limited to, the HKL2000 package (Otwinowski and Minor (1997) “Processing of X-ray Diffraction Data Collected in Oscillation Mode” Methods in Enzymology 276:307-326), the CCP4 package (Collaborative Computational Project (1994) “The CCP4 suite: programs for protein crystallography” Acta Crystallogr D 50:760-763), SOLVE and RESOLVE (Terwilliger and Berendzen (1999) Acta Crystaliogr D 55 (Pt 4):849-861), SHELXS and SIELXD (Schneider and Sheldrick (2002) “Substructure solution with SHELXD” Acta Crystallogr D Biol Crystallogr 58:1772-1779), Refmac5 (Murshudov, et al. (1997) “Refinement of Macromolecular Structures by the Maximum-Likelihood Method” Acta Crystallogr D 53:240-255), PRODRG (van Aalten, et al. (1996) “PRODRG, a program for generating molecular topologies and unique molecular descriptors from coordinates of small molecules” J Comput Aided Mol Des 10:255-262), and 0 (Jones, et al. (1991) “Improved methods for building protein models in electron density maps and the location of errors in these models” Acta Crystallogr A 47 (Pt 2):110-119).

Techniques for structure determination by NMR spectroscopy that can be used to aid in design of polymerases to be screened/selected with the methods herein are similarly well described in the literature. See, e.g., Cavanagh, et al. (1995) Protein NMR Spectroscopy: Principles and Practice, Academic Press; Levitt (2001) Spin Dynamics: Basics of Nuclear Magnetic Resonance, John Wiley & Sons, Evans (1995) Biomolecular NMR Spectroscopy, Oxford University Press; Wuthrich (1986) NMR of Proteins and Nucleic Acids (Baker Lecture Series), Kurt Wiley-Interscience; Neuhaus and Williamson (2000) The Nuclear Overhauser Effect in Structural and Conformational Analysis, 2nd Edition, Wiley-VCH; Macomber (1998) A Complete Introduction to Modem NMR Spectroscopy, Wiley-Interscience; Downing (2004) Protein NMR Techniques (Methods in Molecular Biology), 2nd edition, Humana Press; Clore and Gronenborn (1994) NMR of Proteins (Topics in Molecular and Structural Biology), CRC Press; Reid (1997) Protein NMR Techniques, Humana Press; Krishna and Berliner (2003) Protein NMR for the Millenium (Biological Magnetic Resonance), Kluwer Academic Publishers; Kiihne and De Groot (2001) Perspectives on Solid State NMR in Biology (Focus on Structural Biology, 1), Kluwer Academic Publishers; Jones, et al. (1993) Spectroscopic Methods and Analyses: NMR, Mass Spectrometry, and Related Techniques (Methods in Molecular Biology, Vol. 17), Humana Press; Goto and Kay (2000) Curr. Opin. Struct. Biol. 10:585; Gardner (1998) Annu. Rev. Biophys. Biomol. Struct. 27:357; Wüthrich (2003) Angew. Chem. Int. Ed. 42:3340; Bax (1994) Curr. Opin. Struct. Biol. 4:738; Pervushin, et al. (1997) Proc. Natl. Acad. Sci. U.S.A. 94:12366; Flaux, et al. (2002) Nature 418:207; Fernandez and Wider (2003) Curr. Opin. Struct. Biol. 13:570; Ellman, et al. (1992) J. Am. Chem. Soc. 114:7959; Wider (2000) BioTechniques 29:1278-1294; Pellecchia, et al. (2002) Nature Rev. Drug Discov. (2002) 1:211-219; Arora and Tamm (2001) Curr. Opin. Struct. Biol. 11: 540-547; Flaux, et al. (2002) Nature 418:207-211; Pellecchia, et al. (2001) J. Am. Chem. Soc. 123:4633-4634; and Pervushin, et al. (1997) Proc. Natl. Acad. Sci. USA 94:12366-12371.

The structure of a given polymerase can, as noted, be directly determined, e.g., by x-ray crystallography or NMR spectroscopy, or the structure can be modeled based on the structure of the polymerase. The region of interest of a polymerase can be identified, for example, by homology with other polymerases, examination of various polymerase complexes, biochemical analysis of mutant polymerases, and/or the like. Again, such information can be used to aid in design of mutant polymerases to be screened/selected with the methods herein.

Such modeling of the polymerase can involve simple visual inspection of a model of the polymerase, for example, using molecular graphics software such as the PyMOL viewer (open source, freely available on the World Wide Web at www.pymol.org) or Insight II (commercially available from Accelrys at (www.accelrys.com/products/insight)). Alternatively, modeling of the polymerase or a mutant polymerase, for example, can involve computer-assisted docking, molecular dynamics, free energy minimization, and/or like calculations. Such modeling techniques have been well described in the literature; see, e.g., Babine and Abdel-Meguid (eds.) (2004) Protein Crystallography in Drug Design, Wiley-VCH, Weinheim; Lyne (2002) “Structure-based virtual screening: An overview” Drug Discov. Today 7:1047-1055; Molecular Modeling for Beginners, at (www.usm.maine.edu/˜rhodes/SPVTut/index.html); and Methods for Protein Simulations and Drug Design at (www.dddc.ac.cn/embo04); and references therein. Software to facilitate such modeling is, widely available, for example, the CHARMm simulation package, available academically from Harvard University or commercially from Accelrys (at www.accelrys.com), the Discover simulation package (included in Insight II, supra), and Dynama (available at (www.cs.gsu.edu/˜cscrwh/progs/progs.html). See also an extensive list of modeling software at (www.netsci.org/Resources/Software/Modeling MMMD/top.html).

Visual inspection and/or computational analysis of a polymerase model can identify relevant features of regions of interest, including, for example, amino acid residues of domains that are in close proximity to one another (e.g., those that stabilize inter-domain interactions), residues in the active site that interact with a nucleotide or analogue, or that modulate how large a binding pocket for an analogue is relative to the analogue, etc. A residue can, for example, be deleted or replaced with a residue having a different (smaller, larger, ionic, non-ionic, etc.) side chain or with one that has the ability to bind with, e.g., one or more regions of a nucleotide analogue, a fluorophore, etc.

### Applications for Identified Polymerases

Polymerases identified by the invention are optionally used to copy a template nucleic acid. That is, a mixture of the identified polymerase, nucleotide analogues, and optionally natural nucleotides and other reagents, the template and a replication initiating moiety is reacted such that the polymerase extends an initiating moiety in a template-dependent manner. The moiety can be a standard oligonucleotide primer, or, alternatively, a component of the template, e.g., the template can be a self-priming single stranded DNA, a nicked double stranded DNA, or the like. Similarly, a terminal protein can serve as a initiating moiety. At least one nucleotide analogue can be incorporated into the DNA. The template DNA can be a linear or circular DNA, and in certain applications, is desirably a circular template (e.g., for rolling circle replication or for sequencing of circular templates). Optionally, the composition can be present in an automated DNA replication and/or sequencing system, such as ZMW sequencing applications.

Incorporation of labeled nucleotides by the polymerases identified by the invention can be useful in a variety of different nucleic acid analyses, including real-time monitoring of DNA polymerization. The label can itself be incorporated, or more preferably, can be released during incorporation. For example, incorporation can be monitored in real-time by monitoring label release during incorporation of the nucleotide by the polymerase.

In general, label incorporation or release can be used to indicate the presence and composition of a growing nucleic acid strand, e.g., providing evidence of template replication/amplification and/or sequence of the template. Signaling from the incorporation can be the result of detecting labeling groups that are liberated from the incorporated nucleotide, e.g., in a solid phase assay, or can arise upon the incorporation reaction. For example, in the case of FRET labels where a bound label is quenched and a free label is not, release of a label group from the incorporated nucleotide can give rise to a fluorescent signal. Alternatively, the polymerase enzyme can be labeled with one member of a FRET pair proximal to the active site, and incorporation of a nucleotide bearing the other member will thus allow energy transfer upon incorporation. The use of enzyme bound FRET components in nucleic acid sequencing applications is described, e.g., in Published U.S. Patent application No. 2003-0044781, incorporated herein by reference. It will be appreciated that various polymerases identified through the methods herein can optionally be used in such FRET-sequencing applications.

As described above, in one exemplary sequencing reaction of interest, a polymerase reaction can be isolated within an extremely small observation volume that effectively results in observation of individual polymerase molecules. As a result, the incorporation event provides observation of an incorporating nucleotide that is readily distinguishable from non-incorporated nucleotides. In some aspects, such small observation volumes are provided by immobilizing the polymerase enzyme within an optical confinement, such as a Zero Mode Waveguide. For a description of ZMWs and their application in single molecule analyses, and particularly nucleic acid sequencing, see, e.g., Published U.S. Patent Application No. 2003/0044781, and U.S. Pat. No. 6,917,726, each of which is incorporated herein by reference in its entirety for all purposes. Here too it should be appreciated that polymerases identified through the methods herein can optionally be used in such applications.

In general, for single molecule sequence use of the polymerases identified herein, a polymerase enzyme is complexed with a template strand in the presence of one or more nucleotides. For example, in certain uses, labeled nucleotides are present for each of the four natural nucleotides, A, T, G and C, e.g., in separate polymerase reactions, as in classical Sanger sequencing, or multiplexed together in a single reaction, as in multiplexed sequencing approaches. When a particular base in the template strand is encountered by the polymerase during the polymerization reaction, it complexes with an available labeled nucleotide that is complementary to such nucleotide, and incorporates such into the nascent and growing nucleic acid strand. In one aspect, incorporation can result in a label being released, e.g., in polyphosphate analogues, cleaving between the α and β phosphorus atoms, and consequently releasing the labeling group (or a portion thereof). The incorporation event is detected, either by virtue of a longer presence of the labeled nucleotide and, thus, the label, in the complex, or by virtue of release of the label group into the surrounding medium. Where different labeling groups are used for each of the types of nucleotides, e.g., A, T, G or C, identification of a label of an incorporated nucleotide allows identification of such and consequently, determination of the complementary nucleotide in the template strand being processed at that time. Sequential reaction and monitoring allows for real-time monitoring of the polymerization reaction and determination of the sequence of the template nucleic acid. As noted above, in some aspects, the polymerase enzyme/template complex is provided immobilized within an optical confinement that permits observation of an individual complex, e.g., a Zero Mode Waveguide.

Further details regarding sequencing, PCR, and nucleic acid amplification can be found in Sambrook, Ausubel, Kaufman, Berger, and Rapley, supra, as well as in PCR Protocols A Guide to Methods and Applications (Innis, et al. eds) Academic Press Inc. San Diego, Calif. (1990) (Innis); Chen, et al. (ed.) PCR Cloning Protocols, Second Edition (Methods in Molecular Biology, volume 192) Humana Press; and in Viljoen, et al. (2005) Molecular Diagnostic PCR Handbook Springer, ISBN 1402034032. Further details regarding Rolling Circle Amplification can be found in Demidov (2002) “Rolling-circle amplification in DNA diagnostics: the power of simplicity,” Expert Rev. Mol. Diagn. 2(6): 89-94; Demidov and Broude (eds.) (2005) DNA Amplification: Current Technologies and Applications. Horizon Bioscience, Wymondham, UK; and Bakht, et al. (2005) “Ligation-mediated rolling-circle amplification-based approaches to single nucleotide polymorphism detection” Expert Review of Molecular Diagnostics, 5(1) 111-116.

### EXAMPLES

The following examples are illustrative, but not limiting, of the methods of the present invention. Other suitable modifications and adaptations of the variety of conditions and parameters normally encountered in enzyme (polymerase) screening/selecting, and which would be apparent to those skilled in the art, are within the spirit and scope of the invention.

### Example 1

### Screening for Photostability of Polymerases

The graph of FIG. 3b illustrates the difference in half-life between polymerases that were not screened through use of the methods of the invention (the two polymerases on the left) and polymerases that were screened (the three polymerases on the right). The Figure illustrates the increased fluor-dependant stability of the polymerases that have been screened through the methods of the invention. See above and, for outline of a similar screening methodology, FIG. 2 (for determination of residence time) and FIG. 3a (for determination of CND).

Each polymerase in the current example was generated at 0.2 mM in ACES pH 7.1 buffer containing: 75 mM potassium acetate; 5 mM DTT; and 0.8 uM 24 base oligonucleotide with a 3′ hydroxyl-linked Oregon Green dye (the oligonucleotide in each sample is hybridized to a 72 base template); and 0.05% Tween 20.

The samples in the example were placed in a clear polycarbonate 96-well plate which was placed over an LED light source to excite the dye molecule. Aliquots from the reaction mix were removed prior to the addition of light and after addition of light at 5 minute intervals up to 40-50 minutes.

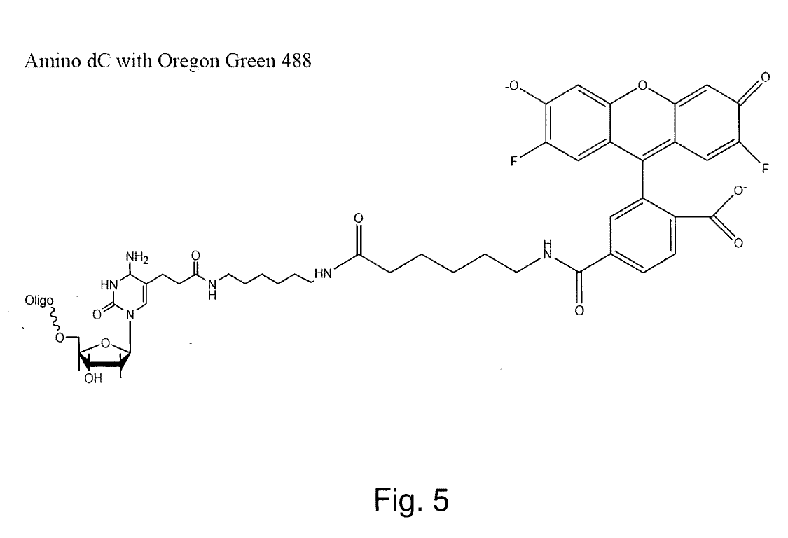

At completion of the light exposure, each sample was then tested for remaining polymerase activity by addition of 0.8 uM of an unlabeled template-primer pair and enzymatic activity was assayed by further addition of 10-15 uM coumarin-labeled dNTPs, 3 mM MnCl₂ and 0.04 u/ml shrimp alkaline phosphatase (SAP). Reactions were monitored through increase of fluorescence of the released coumarin as it was cleaved from the dNTP upon incorporation of the nucleotides by the polymerase. Rate of incorporation was used to estimate remaining polymerase activity after light exposure in the presence of dye. Half-lives of the polymerases were calculated and compared between various variants tested (shown on the graph). As can be seen from the graph, the polymerases that were identified through screening methods of the invention had longer half-lifes, i.e., had increased fluor-dependant stability. FIG. 5 shows the structure of an oligonucleotide with a dye (Oregon Green 488, an amino dC dye) covalently tethered to the terminal base.

### Example II

### Determination of Rates of Release of Cognate Base Pairing (Cognate Nucleotide Dissociation)

Example II illustrates screening of polymerases for decreased cognate nucleotide dissociation or “branching fraction” such is as illustrated herein. In this example, each polymerase sample was generated at 130 mM in 10 mM Tris.HCl pH 7.5 buffer containing: 50 mM potassium acetate; 5 mM DTT; 20 mM ammonium sulfate; 0.05% Tween 20; 0.09% TritonX100; 1 mM calcium chloride; 40 nM hybridized template:primer containing the templating base of interest at the first and second incorporation positions only; and 10 uM analog base of interest (cognate pair to template position 1 and 2). Each polymerase sample was mixed and had added to it: MnCl₂ to 20 mM; 500 uM 3′aminddNTP; and 0.8 uM trap oligonucleotide. The mixtures were then incubated for 5 minutes and the reactions were terminated with 200 uM EDTA.

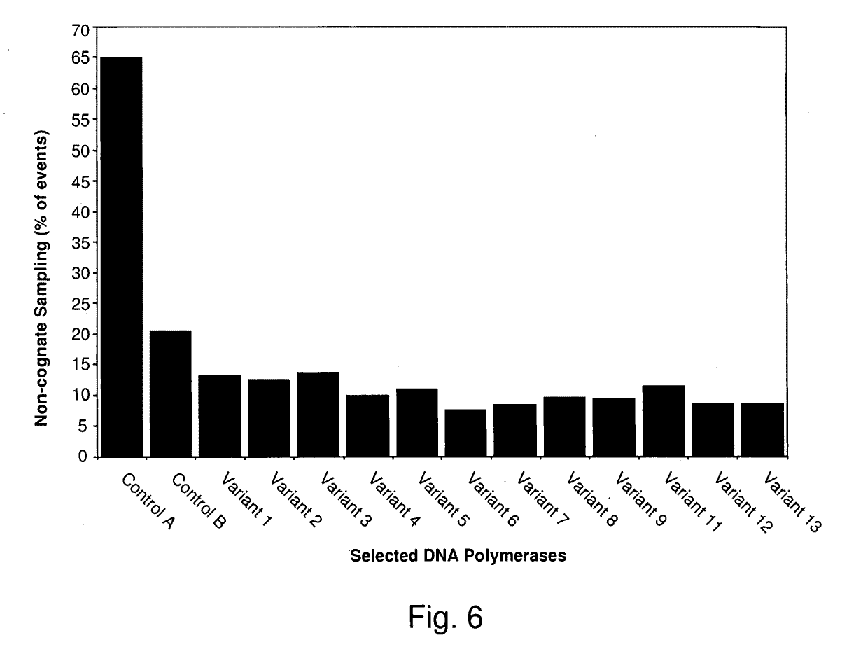

The “cognate nucleotide dissociation” or “branching fraction” was derived by analysis of proportion of +1 product generated as a proportion of summed +1 and +2 product generated (i.e., proportion incorporating only the 3′aminddNTP in competition with the matching analog base). Analysis of reaction products could be performed by acrylamide gel analysis or by capillary electrophoresis and measurement of amount of each product form. FIG. 6 displays a graph showing the amount of noncognate sample (as a percentage of events) of selected polymerases that had been screened through the methods of the invention.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. A method of identifying an improved nucleic acid polymerase having one or more improved characteristics for single molecule sequencing, the method comprising:
providing one or more potentially improved polymerases;
screening and/or selecting the potentially improved polymerases for the one or more improved characteristics; and,
identifying the improved polymerase based on the screening and/or selecting;
wherein the one or more improved characteristics of the polymerase are selected from: increased fluorophore-dependent photostability; increased fluorophore-independent photostability; increased residence time; increased affinity; use of nontraditional divalent cations; decreased cognate nucleotide disassociation activity; increased fidelity; and decreased exonuclease activity, and,
wherein selecting and/or screening includes one or more of: polymerase extension activity in the presence of a fluorescently labeled nucleotide and light; polymerase extension activity in the absence of a fluorescently labeled nucleotide and light; rate of incorporation of a marker nucleotide by a polymerase; incorporation of a marker nucleotide by polymerase extension activity under limiting concentrations of nucleotides; rate of incorporation of a marker nucleotide by a polymerase in the presence of nontraditional divalent cations; rate of cognate nucleotide disassociation; and removal of a marker nucleotide from a nucleic acid by a polymerase.

2. The method of claim 1, wherein the potentially improved polymerases comprise one or more randomly mutated nucleic acid polymerases and/or one or more rationally designed mutated nucleic acid polymerases.

3. The method of claim 1, wherein the potentially improved polymerase is selected and/or screened for viable polymerase activity before, or at the same time, it is selected and/or screened for the one or more improved characteristics.

4. The method of claim 1, wherein the improved characteristic comprises increased fluorophore-dependent photostability and wherein the screening and/or selecting comprises polymerase extension activity in the presence of a fluorescently labeled nucleotide and light.

5. The method of claim 4, further comprising providing one or more fluorophore-labeled oligonucleotide hybridized to a nucleic acid template that is acted upon by the polymerase.

6. The method of claim of claim 5, wherein the fluorophore is located at the 3′ end of the oligonucleotide.

7. The method of claim 6, wherein the fluorophore is in close proximity to the binding site of the polymerase wherein the polymerase interacts with the nucleic acid template.

8. The method of claim 1, wherein the improved characteristic comprises increased fluorophore-independent photostability and wherein the screening and/or selecting comprises polymerase extension activity in the presence of light and in the absence of a fluorescently labeled nucleotide.

9. The method of claim 1, wherein the improved characteristic comprises increased residence time and wherein the screening and/or selecting comprises rate of incorporation of a marker nucleotide by a polymerase.

10. The method of claim 1, wherein the improved characteristic comprises increased affinity and wherein the screening and/or selecting comprises incorporation of a marker nucleotide by polymerase extension activity under limiting concentrations of nucleotides.

11. The method of claim 1, wherein the improved characteristic comprises use of nontraditional divalent cations and wherein the screening and/or selecting comprises rate of incorporation of a marker nucleotide by a polymerase in the presence of nontraditional divalent cations.

12. The method of claim 1, wherein the improved characteristic comprises decreased cognate nucleotide disassociation activity and wherein the screening and/or selecting comprises rate of cognate nucleotide disassociation.

13. The method of claim 1, wherein the improved characteristic comprises increased fidelity and wherein the screening and/or selecting comprises rate of incorporation of a non-cognate nucleotide.

14. The method of claim 1, wherein the improved characteristic comprises decreased exonuclease activity and wherein the screening and/or selecting comprises removal of a marker nucleotide or exposure/activation of a marker nucleotide from a nucleic acid by a polymerase.

15. The method of claim 1 wherein a polymerase activity of the one or more potentially improved polymerases and/or the improved polymerase is determined by oligonucleotide probe hybridization.

16. The method of claim 1, wherein the one or more potentially improved polymerases and/or the improved polymerase are tracked by DNA identification tagging.

17. The method of claim 1, wherein the one or more improved characteristics comprises a first characteristic and at least a second characteristic.

18. The method of claim 13, wherein the first characteristic and the at least second characteristic are screened and/or selected for simultaneously.

19. The method of claim 13, wherein the first characteristic and the at least second characteristic are screened and/or selected for sequentially.

20. The method of claim 1, wherein screening and/or selecting comprises a first screening and/or selecting and at least a second screening and/or selecting.

21. The method of claim 16, wherein the first screening and/or selecting and the at least second screening and/or selecting are performed simultaneously.

22. The method of claim 16, wherein the first screening and/or selecting and the at least second screening and/or selecting are performed sequentially.

23. The method of claim of claim 16, wherein the first screening and/or selecting and the at least second screening and/or selecting are for different improved characteristics.

24. The method of claim 18, wherein the first screening and/or selecting and the at least second screening and/or selecting are for the same improved characteristic.

25. A polymerase chosen by the method of claim 1.

26. A system of identifying putative improved polymerases, the system comprising
a screening module configured to perform one or more of:
polymerase extension activity in the presence of a fluorescently labeled nucleotide and light;
polymerase extension activity in the absence of a fluorescently labeled nucleotide and light;
rate of incorporation of a marker nucleotide by a polymerase; incorporation of a marker nucleotide by polymerase extension activity under limiting concentrations of nucleotides; rate of incorporation of a marker nucleotide by a polymerase in the presence of nontraditional divalent cations; rate of cognate nucleotide disassociation; and removal of a marker nucleotide from a nucleic acid by a polymerase; and,
a detector configured to detect one or more improved characteristic selected from:
increased fluorophore-dependent photostability; increased fluorophore-independent photostability; increased residence time; increased affinity; use of nontraditional divalent cations; decreased cognate nucleotide disassociation activity; increased fidelity; and decreased exonuclease activity.
